(19) United States
(12) Patent Application Publication
(10) Pub. No.: US 2023/0248628 A1
(43) Pub. Date: Aug. 10, 2023

(54) COMPOSITION FOR HAIR GROWTH AND/OR HAIR RESTORATION

(71) Applicant: Institute of Rheological Function of Food Co., Ltd., Fukuoka (JP)

(72) Inventors: Takehiko FUJINO, Fukuoka (JP); Shiro MAWATARI, Fukuoka (JP); Masanori HONSHO, Fukuoka (JP)

(73) Assignee: Institute of Rheological Function of Food Co., Ltd., Fukuoka (JP)

(21) Appl. No.: 18/010,587

(22) PCT Filed: Jun. 28, 2021
(86) PCT No.: PCT/JP2021/024299

§ 371 Date: Dec. 15, 2022

(30) Foreign Application Priority Data
Jun. 30, 2020 (JP) 2020-113073
Mar. 4, 2021 (JP) 2021-034106

(51) Int. Cl. A61K 8/55; A61Q 7/00

(52) U.S. Cl. CPC A61K 8/55; A61Q 7/00

(57) ABSTRACT

A composition having hair growth and/hair restoration effect that includes plasmalogen extracted from tissues of animal such as shellfish, sea squirt, and birds, and that can be used as external preparation or oral preparation.

### TECHNICAL FIELD

The present invention relates to a composition that promotes hair growth and/or hair restoration.

### BACKGROUND ART

Plasmalogen is one type of phospholipid having an antioxidant effect, and is one of glycerophospholipid. Plasmalogen is present in all tissues of mammals, and represents about 18% of phospholipid in human body. However, it is known to be particularly abundant in cranial nerve, cardiac muscle, skeletal muscle, leucocytes, and sperm.

Plasmalogen is known for its action of promoting neurogenesis, action of suppressing nerve inflammation due to lipopolysaccharides (LPS), action of suppressing accumulation of amyloid β (Aβ) protein in brain, etc., and it is said to have effect on cranial nerve disorders such as Alzheimer’s disease, Parkinson’s disease, depression, and schizophrenia. For example, in non-patent reference 1, it is reported that in patients having orally administered scallop derived-purified plasmalogen, memory function of mild Alzheimer’s disease are ameliorated.

On the other hand, recently, thin hair is becoming a serious problem not only in men, but also in women. Hair consists of hair shaft being outside of scalp, and hair root inside the scalp. In hair bulb part being the base end section of hair root, hair matrix cells forming the hair, and hair papilla cells that control the action of hair matrix cells are present. The hair root is fixed to the scalp by the root sheath consisting of internal root sheath and external root sheath. The external root sheath has a bulge region where hair follicle stem cells and melanocyte stem cells are present, and since it comprises “CD34 positive cells” being the base of hair matrix cells, it is thought to be one of essential factors for generating hair.

Further, it is thought to be critical to promote growth of hair matrix cells that generate hair shaft and root sheath cells around hair root to resolve thin hair.

Further it is known that when neovascular vessels increase in scalp, it is possible to efficiently provide nutrition to hair root, and thus hair growth and/or hair restoration is promoted.

Under such circumstances as in the above, almost no study of the influence of plasmalogen on hair growth and/or hair restoration has been made up to now.

### CITATION LIST

### Non-Patent Literature

Non-Patent Literature 1: Fujino T.et al, “Efficacy and Blood Plasmalogen Changes by Oral Administration of Plasmalogen in Patients with Mild Alzheimer’s Disease and Mild Cognitive Impairment: A Multicenter, Randomized, Double-blind, Placebo-controlled Trial” EBioMedicine, [17] (2017) 199-205

### SUMMARY OF INVENTION

### Technical Problem

The object of the present invention is to provide a composition having an excellent hair growth and/or hair restoration effect.

### Solution to Problem

The present inventors made a keen study to solve the above-mentioned object, and as a result, they found out that plasmalogen promotes phosphorylation of AMPK (AMP- activated protein kinase) of human adult hair outer root sheath cells, or promote expression of VEGF (vascular endothelial growth factor) of human fibroblast cells, and exerts an excellent effect on hair growth and/or hair restoration. The present invention has been thus completed.

Specifically, the present invention relates to the following.

A composition for hair growth and/or hair restoration, comprising plasmalogen.

The composition for hair growth and/or hair restoration according to [1], wherein the plasmalogen is a plasmalogen extracted from an animal tissue.

The composition for hair growth and/or hair restoration according to [2], wherein the animal tissue is a tissue of an animal selected from shellfish, sea squirt, and birds.

The composition for hair growth and/or hair restoration according to [2] or [3], wherein the animal tissue is a tissue of scallops.

The composition for hair growth and/or hair restoration according to any one of [1] to [4], wherein the plasmalogen is an ethanolamine plasmalogen.

### Advantageous Effects of Invention

The composition of the present invention has an excellent hair growth/hair restoration effect.

### BRIEF DESCRIPTION OF DRAWINGS

FIG. 1 shows the results of promotion of phosphorylation of AMPK (relative amount of Phospho-AMPK with respect to AMPK) in HHORSC cells (human hair outer root sheath cells) by the treatment with scallop derived-plasmalogen (0.5 µg/mL, 3 hours).

FIG. 2 shows the results of promotion of phosphorylation of AMPK (relative amount of Phospho-AMPK with respect to AMPK) in HHORSC cells (human hair outer root sheath cells) by the treatment with scallop derived-plasmalogen (5 µg/mL, 7 hours).

FIG. 3 shows the results of VEGF expression in HDF-a cells (human fibroblast cells) by the treatment with scallop derived-plasmalogen (5 µg/mL, 7 hours).

FIG. 4 shows results of VEGF expression in HDF-a cells (human fibroblast cells), by the treatment of plasmalogen (5 µg/mL, 7 hours) derived from various animals.

FIG. 5 shows photographs showing the results of hair growth (representative examples of after 1 week and of after 2 weeks after shaving hair) of the shaved part of mouse applied with plasmalogen solution.

FIG. 6 shows microphotographs of hair root by Hematoxylin-Eosin staining of mouse skin applied with plasmalogen for 4 weeks.

### DESCRIPTION OF EMBODIMENTS

The composition for hair growth and/or hair restoration of the present invention is characterized by comprising plasmalogen.

The composition of the present invention promotes phosphorylation of AMPK of human hair outer root sheath cells or VEGF expression of human fibroblast cells, and exerts an excellent effect on hair growth and/or hair restoration. Outer root sheath cells play an important role in hair growth and/or hair restoration, and by activating the same, it is possible to promote hair growth and/or hair restoration. Further, as VEGF secreted in fibroblast cells in scalp act on vascular endothelial cells, neovascular cells increase and by efficiently providing nourishment to hair root, it is possible to promote hair growth and/or hair restoration.

Specifically, the composition comprising plasmalogen of the present invention can be used as a composition for activating AMPK of outer root sheath cells, a composition for promoting VEGF expression, a composition for promoting vascularization, or a composition for hair growth and/or hair restoration. Here, hair growth in the present invention relates to grow hair which has been lost, such as treatment of patients with alopecia, and hair restoration relates to make the present hair to be strong hair, such as preventing of thin hair or hair loss.

Plasmalogen used in the present invention is one type of phospholipid having an antioxidant effect, and is one of glycerophospholipid. It is a unique subclass of glycerophospholipid characterized by having a vinyl-ether linkage in the sn-1 position of glycerol backbone. It is observed at a high concentration in cell membrane in tissues of many mammals. As plasmalogen, those having a fatty acid ester linkage in the sn-2 position is preferable.

The plasmalogen used in the present invention is not particularly limited as long it is generally classified as plasmalogen, and examples include choline plasmalogen, ethanolamine plasmalogen, inositol plasmalogen, and serine plasmalogen. Among these, choline plasmalogen and ethanolamine plasmalogen are preferable, and ethanolamine plasmalogen is particularly preferable.

The plasmalogen of the present invention can be extracted from animal tissues. Animal tissues are not particularly limited as long it comprises plasmalogen, and examples include aquatic animals such as shellfish, sea squirt, sea cucumber, salmon, skipper and bonito, and birds. Among these, shellfish, sea squirt, and birds are preferable, and shellfish are particularly preferable. As parts to be used, edible part (part that can be eat) is preferable. These animal tissues can be cut products, but it is preferable to use ground products since plasmalogen can be extracted more efficiently.

Examples of shellfish include edible clams such as scallops, mussels, and abalone, and snails, and scallops are particularly preferable. Scallops are edible clams belonging to Pectinidae, and for example, those belonging to the genus Mizuhopecten, and the genus Pecten can be exemplified. Specifically, common scallop (scientific name: Mizuhopecten yessoensis) collected in Japan, or European scallop (scientific name: Pecten maximus (Linnaeus) ) collected in Europe can be exemplified. As edible parts, scallop eye and strings can be exemplified.

Sea squirts are edible chordates belonging to Pyuridaethe, and those belonging to the genus Haloncynthia, the genus Halocynthia aurantium can be exemplified. Specifically, Maboya (scientific name: Haloncynthia roretzi) and Akaboya (scientific name: Halocynthia aurantium) can be exemplified. As edible parts, meats (fascia) can be exemplified.

Birds are not particularly limited as long as it is edible birds, and for example, chicken, silky fowl and canard can be exemplified. As edible parts, breast meat comprising plasmalogen in abundance is preferable.

Extraction of plasmalogen can be performed by using water, organic solvent, and water-containing organic solvent, and it is preferable to perform enzyme treatment in combination. For example, ethanol extraction method, and hexane extraction method can be exemplified, and ethanol extraction method is preferable.

Ethanol extraction method is not particularly limited as long it is a method of extracting using ethanol (including water-containing ethanol), and examples include methods described in Japanese published unexamined application No. 2019-140919, Japanese published unexamined application No. 2018-130130, Republished patent No. 2012-039472, Japanese published unexamined application No. 2010-065167, and Japanese published unexamined application No. 2010-063406, etc.

Hexane extraction method is not particularly limited as long as it is a method of extracting using hexane, and examples include methods described in Republished patent No. 2009-154309, Republished Patent No. 2008-146942, etc.

The composition of the present invention can be used as an oral agent or a parenteral agent. As for a parental agent, examples include external agent and injection agent. The external agent is not particularly limited as long as it can applied to scalp. Example of its form include skin external agent such as ointment, cream, gel, lotion, emulsion, pack, poultice, etc. Specifically, those that can be commonly used for hair growth and/or hair restoration, such as hair tonic, shampoo, rinse, pomade, hair lotion, hair cream, hair treatment, etc. can be exemplified.

Further, when using as an oral agent, examples of its form include, for example, tablet form, capsule form, powder form, granule form, liquid form, grain form, bar form, plate form, block form, solid form, pellet form, paste form, cream form, caplet form, gel form, chewable form, stick form, or the like. Among these, capsule form is preferable.

The composition for hair growth and/or hair restoration of the present invention is not particularly limited as long as it can be distinguished from other products as product, in the point of comprising plasmalogen, and being used for hair growth and/or hair restoration, and examples include medicine (including quasi-drug), cosmetics, or as so-called health food products such as functional foods which indication of efficacy is allowed from a prescribed authority, including foods for specified health use, foods with nutrient function claims, foods with function claims, or the like. Further, those with an indication of having hair growth and/or hair restoration effect on any of the main body, package, instructions, advertisement of the product of the present invention are encompassed in the scope of the present invention.

For example, in cosmetics or health food products, it can be indicated specifically as “grow hair”, “stimulate hair growth”, “stimulate growth of hair”, “prevent thin hair”, “to those who are worried of thin hair”, “to those who are worried of hair loss”, or the like.

As for the content of plasmalogen in the composition of the present invention, it can be appropriately comprised within the scope with which the effect is exerted. It depends on the form, but for example it is preferable that plasmalogen is 10⁻¹⁰% by mass or more of the whole composition of the present invention, in terms of dry mass equivalent, more preferable to be 10⁻⁵% by mass or more, further preferable to be 0.1% by mass or more, and particularly preferable to be 1.0% by mass or more.

The amount of intake in case where the composition of the present invention is an oral agent is not particularly limited. However, from the viewpoint to more significantly exert the effect of the present invention, it is preferable to intake so that the amount of intake of plasmalogen of an adult per day is 10⁻⁶ µg or more per day, more preferable so that it is 1 µg or more per day, further preferable so that it is 500 µg or more per day, and particularly preferable so that it is 1000 µg or more per day. The upper limit is for example 20,000 µg per day, and preferably 10,000 µg per day.

The composition of the present invention can be stored in one container, or for example in plural containers of 2 to 3, so that the amount of intake per day becomes the above-mentioned amount of intake, for one day.

The composition of the present invention can be produced by known production methods by adding ingredients other than active ingredients (plasmalogen) acceptable as oral agent, external agent or injection agent, according to need.

Examples of other ingredients other than active ingredients of the present invention include, for example, vitamin, mineral, protein, peptide, amino acid, animal oil, vegetable oil. Further, various ingredients according to diverse purposes, such as hydrocarbons, waxes, oil and fat, esters, higher fatty acids, higher alcohol, surfactant, fragrance, pigment, antiseptic, antioxidant, ultraviolet protectant, alcohols, pH adjuster, etc. applied to hair growth and/or hair restoration, or the like can be mixed.

In the following, the present invention will be explained in detail, based on Examples.

### Example 1

The effect of promoting phosphorylation of AMPK in HHORSC cells (human hair outer root sheath cells) by plasmalogen, active ingredient of the composition of the present invention, was confirmed.

### Plasmalogen

As plasmalogen, ethanolamine plasmalogen (PlsEtn) prepared by extracting common scallop (scientific name: Mizuhopecten yessoensis) with ethanol, and purified with HPLC was used.

### Cell Culture

As human hair outer root sheath cells, Human Hair Outer Root Sheath (HHORSC) cells (#2420) purchased from ScienCell Research Laboratories were used. HHORSC cells were cultured in Mesenchymal Stem Cell Medium (MSCM, #7501) . Cells up to 10 passages were used in the experiment.

HHORSC cells cultured from the previous day were cultured for a predetermined time (3 hours or 7 hours) in a mixed medium of 900 µL of FM and 100 µL of PLsEtn solution in which PlsEtn (0.5 µg or 5 µg) was previously suspended by ultrasonic treatment in Opti-MEM™ Reduced Serum Medium (ThemoFisher, #22600050).

### Analysis of Phosphorylation of AMPK

HHORSC cells were collected with Buffer A (0.25 M sucrose, 10 mM Hepes-KOH, pH 7.5, 1 mM EDTA, protease inhibitor cocktail) and centrifuged. The obtained cells were suspended in Buffer A, crushed by ultrasonic treatment, and after protein determination, the same protein amount was subjected to electrophoresis. Next, the resultant was transferred on PVDF membrane, and detected by western blotting using Phospho-AMPK α (Thr172) antibody (Cell Signaling technology, #2535S) and AMPK α antibody (Cell Signaling technology, #58315). The signal was determined by Multi Gauge software version 3.0 software (Fuji Film). The signal obtained with Phospho-AMPK α (Thr172) antibody was divided by the signal obtained with AMPK α antibody for standardization. Further, the level obtained from untreated cells was set as 1, to show the signal strength in cells having undergone each treatment as a relative value. It was tried 3 times or more, and the mean level and standard deviation were shown.

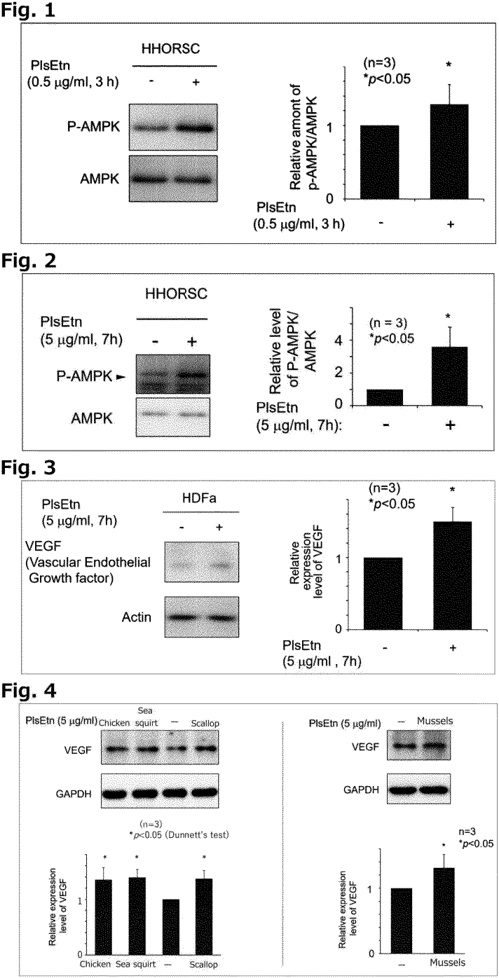

FIG. 1 shows the results of effect of promoting phosphorylation of AMPK (relative amount of Phospho-AMPK with respect to AMPK) in HHORSC cells which were cultured for 3 hours in the presence of 0.5 µl of PlsEtn. Further, FIG. 2 shows the results of effect of promoting phosphorylation of AMPK (relative amount of Phospho-AMPK with respect to AMPK) in HHORSC cells which were cultured for 7 hours in the presence of 5 µg/ml of PlsEtn.

As shown in FIG. 1 and FIG. 2, in HHORSC cells cultured in the presence of PlsEtn, phosphorylation of AMPKα essential for AMPK activation was enhanced as compared to cells cultured in the absence of PlsEtn.

From the above results, it can be thought that PlsEtn promotes phosphorylation of AMPK in HHORSC cells, and can achieve hair growth and/or hair restoration.

### Example 2

Effect of promoting VEGF expression in HDF-a cells (human fibroblast cells) by plasmalogen, active ingredient of the composition of the present invention, was confirmed.

### Plasmalogen

As plasmalogen, ethanolamine plasmalogen (PlsEtn) extracted and purified similarly as Example 1 was used.

### Cell Culture

As human fibroblast cells, Human Dermal Fibroblasts-adult (HDF-a) cells (#2320) were used. HDF-a cells were cultured in Fibroblast Medium (FM, #2301). Cells up to 6 passages were used in the experiment.

HDF-a cells cultured from the previous day were cultured for 7 hours in the presence of 5 µg/ml of PlsEtn.

As comparison, HDF-a cells were similarly cultured in the absence of PlsEtn.

### Analysis of VEGF Expression Promotion

Cultured HDF-a cells were collected with buffer A (0.25 M sucrose, 10 mM Hepes-KOH, pH 7.5, 1 mM EDTA) and centrifuged. The obtained cells were suspended in buffer A, crushed by sonication, and after protein determination, the same amount of protein was subjected to electrophoresis. Then, the resultant was transferred on PVDF membrane, and detected by western blotting using anti-vascular endothelial growth factor (VEGF) antibody (Protein tech #19003-1-AP), and anti-actin antibody (MBL #M177-3). Signal of each obtained protein was determined by using Multi Gauge software version 3.0 software (Fuji Film) and standardized by dividing the VEGF signal by actin signal. Further, the untreated level was set to be 1, and the relative level of VEGF signal strength obtained in each treatment was shown by the difference of the mean level and the mean level of the two trials.

FIG. 3 shows the results of promoting VEGF expression in cultured HDF-a cells (relative level of VEGF with respect to actin).

As shown in FIG. 3, in HDF-a cells cultured in the presence of PlsEtn, VEGF expression was enhanced as compared to cells cultured in the absence of PlsEtn. Therefore, it is thought that PlsEtn prompts vascularization by promoting biosynthesis of VEGF, and can achieve hair growth and/or hair restoration.

### Example 3

Similarly as Example 2, effect of promoting VEGF expression in HDF-a cells (human fibroblast cells) by plasmalogen was confirmed. In this example, as plasmalogen, besides those derived from scallop, plasmalogens derived from chicken (scientific name: Gallus gallus domesticus) breast meat derived, sea squirt (scientific name: Halocynthia roretzi), and mussel (scientific name: Mytilus Linnaeus) were used.

FIG. 4 shows the results of promoting VEGF expression in HDF-a cells cultured in the presence of plasmalogen derived from various animals (relative level of VEGF with respect to GAPDH).

As shown in FIG. 4, similarly as with scallop-derived plasmalogen, with chicken (breast meat)-, sea squirt-, and mussel-derived plasmalogens, VEGF expression was enhanced. Therefore, it is thought that plasmalogen derived from various animals prompts vascularization to promote biosynthesis of VEGF, and can achieve hair growth and/or hair restoration.

### Example 4

Effect of promoting hair growth by plasmalogen was confirmed by using mouse.

### Plasmalogen Solution

1% plasmalogen solution in which mixed plasmalogen (Pls) of 51 mg of Scallop derived-PlsEtn, 5 mg of chicken breast meat-derived ether phospholipid (contained at PL content 9.1%, PlsEtn : PlsCho = 3:4) was dissolved with 5.2 ml of 70% ethanol was used.

### Experimental Animal

As experimental animal, male C3H mouse (7 weeks old) commonly used for evaluating hair growth promotion was used.

### Application of Plasmalogen Solution to Mouse

10 of the above mentioned 7 weeks-old male C3H mice were preliminary bred. Body hair on the dorsal skin was shaved at the age of 8 weeks-old with an electric hair clipper for animals and depilated with depilatory cream. From 48 hours after shaving, application of 70% EtOH solution containing 10 mg/ml Pls was started. Frequency of application was 5 times/week for 2 weeks, and the quantity of application was 20 µl/cm². Further, as control, 70% EtOH solution not containing plasmalogen was applied at the same frequency and quantity of application. There were 5 mice in each treatment section.

### Confirmation of Hair Growth in Mouse

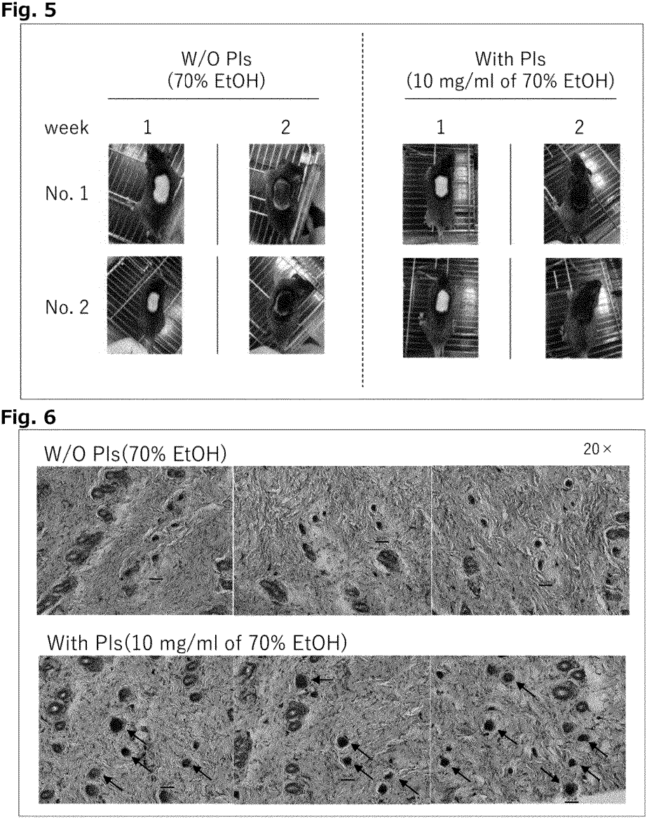

FIG. 5 shows the results of observing shaved part of mice, after 1 week after shaving and after 2 weeks after shaving.

As shown in FIG. 5, after 2 weeks after shaving, on shaved parts of mice applied with solution not containing plasmalogen, skin partially with no hair growth can be confirmed, while on shaved parts of mice applied with solution containing plasmalogen, hair growth is confirmed all around, and an apparent difference in hair growth level was recognized. Specifically, it has been revealed that plasmalogen has an effect of promoting hair growth.

### Example 5

It is known that during the growth period of hair root, number of cells of hair root is increased and overgrown as compared to during resting period. Therefore, the state of hair root of mouse skin applied with plasmalogen for a prescribed time period was confirmed.

Similarly as the method of Example 4, after applying 70% EtOH solution containing 10 mg/ml Pls for 4 weeks to mice, mouse skin piece of the applied part was obtained and subjected to Hematoxylin-Eosin staining. As control, 70% EtOH solution not containing plasmalogen was applied to mice at the same frequency and quantity of application.

FIG. 6 shows microphotographs of hair root by Hematoxylin-Eosin staining of mouse skin applied with plasmalogen for 4 weeks. Scale bar in the figure shows 50 µm.

As shown in FIG. 6, on mouse skin applied with a solution comprising plasmalogen, a larger cell population of hematoxylin positive staining cell nuclei was observed as compared to control, and it is thought that a large number of hair root is in growth period. Therefore, it is thought that plasmalogen promotes cell growth increase of hair root, and can achieve hair growth and/or hair restoration.

### Example 6

### Formulation Example 1

Hair growing agent (100 g) was produced according to the following formulation.

| | |
|---|---|
| Scallop extracted plasmalogen | 0.5 mg |
| Glycerin | 0.5 mg |
| Purified water | remaining part |

### Formulation Example 2

A hard capsule agent was produced according to the following formulation.

| | |
|---|---|
| Scallop extracted plasmalogen | 0.5 mg |
| Cytrodextrin | 3.3 mg |
| Amino acids | 1.2 mg |
| Pine-Dex | 185.0 mg |

### INDUSTRIAL APPLICABILITY

Since the composition of the present invention can be used as an external agent or oral agent, it is industrially useful.

1-2. (canceled)

3. A method of hair growth and/or hair restoration, comprising administering a composition comprising plasmalogen to human.

4. The method according to claim 3, wherein the plasmalogen is a plasmalogen extracted from an animal tissue.
